(19) United States
(12) Patent Application Publication
(10) Pub. No.: US 2007/0241206 A1
(43) Pub. Date: Oct. 18, 2007

(54) Scent delivery device

(76) Inventor: Jacquelyn Lowden Williams, Temecula, CA (US)

Correspondence Address: Gerhard P. Shipley, 300 Clayton Court, Lawrence, KS, 66044, US

(21) Appl. No.: 11/787,806

(22) Filed: Apr. 18, 2007

Related U.S. Application Data
(60) Provisional application No. 60/792,836, filed on Apr. 18, 2006.

(51) Int. Cl. A61L 9/04

(52) U.S. Cl. 239/57; 239/58

(57) ABSTRACT

A scent delivery device (10) for attachment to a door or window screen (22) exposed to a natural airflow providing a vector for dispersing the scent over time. The device (10) includes a body (12) and two or more legs (14a,14b). The body (12) includes a reservoir (16) for containing the scent, and includes an opening (15) to facilitate dispersal of the scent from the reservoir (16). The legs (14a,14b) are connected to and extend outwardly from the body (12). The legs (14a,14b) each include a length of wire having a diameter which is smaller than the mesh of the screen (22) for attaching the body (12) to the screen (22).

### RELATED APPLICATIONS

The present U.S. non-provisional patent application relates to and claims priority benefit of an earlier-filed U.S. provisional patent application of the same title, Ser. No. 60/792,836, filed Apr. 18, 2006. The identified earlier-filed application is incorporated by reference herein.

### BACKGROUND OF THE INVENTION

1. Field of the Invention

The present invention relates broadly to devices for delivering scents. More particularly, the present invention concerns a scent delivery device adapted for physical association with a screen, or mesh, such as attachment to a door screen or window screen, wherein natural airflow through the screen provides a vector for dispersal of the scent.

2. Description of the Prior Art

Scent delivery devices are known that release scent from a reservoir when exposed to air. However, these devices are passive, such that the released scent remains concentrated in relatively close proximity to the devices.

Scent delivery devices are also known that are adapted for attachment to the outlets, or vents, of heating or cooling apparatuses. The artificial airflow generated by the apparatuses disperses the released scent. However, in certain climates and at certain times of the year it is preferable to open doors and windows. As heating and cooling apparatuses are normally turned off when doors and windows are open, the generated artificial airflow ceases and the scent is again concentrated in relatively close proximity to the devices.

Due to these and other problems and disadvantages encountered in the prior art, a need exists for an improved scent delivery device.

### SUMMARY OF THE INVENTION

The present invention overcomes the above-described and other problems and disadvantages by providing a scent delivery device for attachment to a door or window screen exposed to a natural airflow for dispersing a scent over time.

In one embodiment, the device comprises a body including a reservoir for containing the scent, the reservoir being exposed to the natural airflow, and an opening through which the natural airflow can flow; and two or more legs connected to and extending outwardly from the body for attaching the body to the screen. The device may further include a movable panel for controlling a size of the opening. The reservoir may be refillable with the scent. The scent may be in a substantially liquid form. The device may include a substantially solid carrier for receiving and releasing the scent over time into the natural airflow. The two or more legs may each include a length of wire having a diameter which is smaller than the mesh of the screen. At least one of the two or more legs may be bendable so as to positively engage the screen. At least one of the two or more legs may have the shape of a hook to positively engage to the screen. The body may have substantially the shape of an insect or a leaf. The device may further include an absorbent carrier for receiving the scent from the reservoir. The reservoir may be actuatable to force the scent through delivery tubes and into the absorbent carrier.

In a second embodiment, the device broadly comprises the body including an actuatable reservoir for holding the scent, an absorbent carrier for receiving the scent from the reservoir, a delivery tube extending between the actuatable reservoir and the absorbent carrier for delivering the scent from the actuatable reservoir to the absorbent carrier when the actuatable reservoir is actuated, and an opening through which the natural airflow can flow; and two or more legs connected to and extending outwardly from the body for attaching the body to the screen.

From the description set forth herein, it will be appreciated that the scent delivery device of the present invention provides a number of substantial advantages over the prior art, including, for example, allowing for the wide dispersal of scent even when artificial airflow generators, such as heating and cooling apparatuses, have been turned off.

These and other features of the present invention are described in more detail in the section titled DETAILED DESCRIPTION OF THE INVENTION, below.

### BRIEF DESCRIPTION OF THE DRAWINGS

A preferred embodiment of the present invention is described in detail herein with reference to the attached drawing figures, wherein:

FIG. 1 is an isometric view of a first embodiment of the device of the present invention;

FIG. 2 is an isometric view of an end of a first embodiment of a leg component of the device engaging a screen;

FIG. 3 is an isometric view of the end of a second embodiment of the leg component of the device engaging a screen; and

FIG. 4 is a plan view of a second embodiment of the device of the present invention.

### DETAILED DESCRIPTION OF THE INVENTION

With reference to the figures, a scent delivery device 10 is described, shown, and otherwise disclosed herein in accordance with one or more preferred embodiments of the present invention. Broadly, the device 10 is adapted for physical association with a screen, or mesh, such as attachment to a door screen or window screen, wherein natural airflow through the screen provides a vector for dispersing the scent.

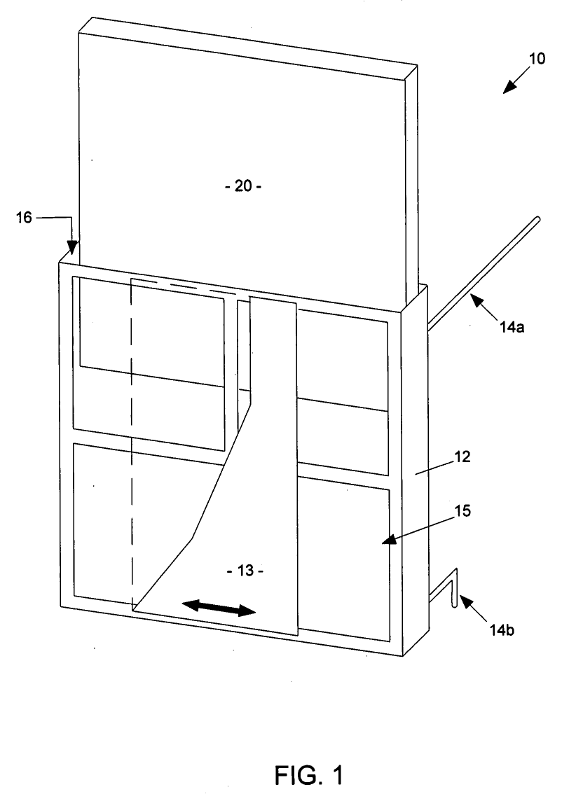

Referring to FIG. 1, a first embodiment of the device 10 broadly comprises a body 12 and two or more legs 14a,14b. The body 12 includes a reservoir 16 for containing the scent. The body 12 may present one or more openings 15 to facilitate dispersal of the scent from the reservoir 16. The body 12 may present a movable panel 13, such as, for example, a sliding door or folding flap, to facilitate controlling the size of the openings 15 and thereby controlling the amount of scent being dispersed into the airflow. The reservoir 16 may be refillable, or rechargeable. The scent may be in a liquid or gel form or embedded, impregnated, or otherwise incorporated into a substantially solid carrier 20, wherefrom the scent is released over time into the airflow.

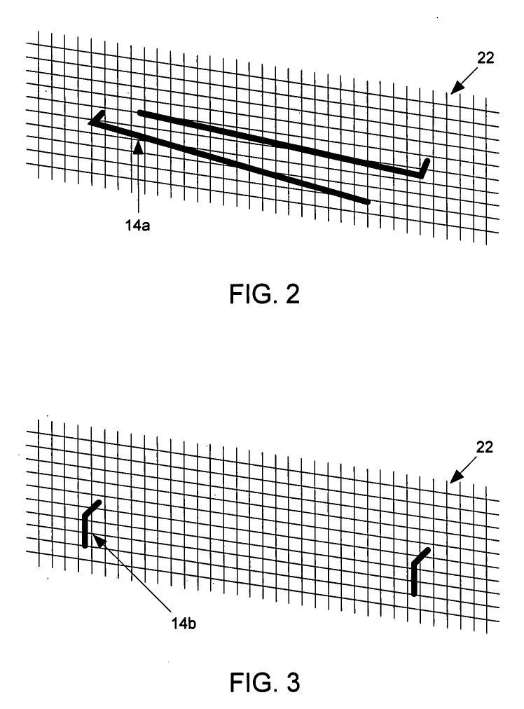

Referring also to FIGS. 2 and 3, the two or more legs 14a,14b are connected to and extend outwardly from the body 12, and facilitate physically attaching the body 12 with a door, window, or other screen 22. There may be between two legs and four legs. As shown in FIG. 2, in a first embodiment of the leg 14a, the leg 14a is substantially bendable to allow for securing the device 10 in position on the screen 22. As shown in FIG. 3, in a second embodiment of the leg 14b, the end of the leg 14b presents a hook or other structure for engaging the screen 22. A particular instance of the device 10 may present only first embodiment legs 14a, only second embodiment legs 14b, or, as shown in FIG. 1, a combination of first and second embodiment legs. The legs 14a,14b are preferably manufactured from metal or plastic wire having a diameter smaller than the mesh of the screen 22.

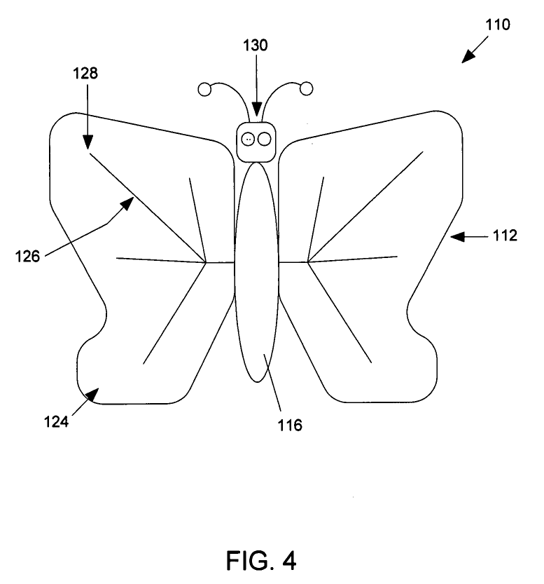

Referring also to FIG. 4, the device 10, particularly the body 12 thereof, may be given an ornamental shape or silhouette, such as that of an insect, e.g., a butterfly, a leaf, or a flower, in order to be more aesthetically appealing.

In exemplary but non-limiting use and operation, the device 10 may function as follows. The two or more legs 14 are pushed through or against the screen 22, and, as necessary, bent, so as to positively engage the screen 22, thereby securely attaching the device 10 to the screen 22. Thereafter, the scent contained in the reservoir 16 is released and dispersed by natural airflow through the screen 22 and through the openings 15 in the body 12. Periodically, the scent may be renewed by, for example, refilling or replacing the reservoir 16.

Referring again to FIG. 4, a second embodiment of the device 110 is shown which may be substantially similar to the first embodiment described above except for the following differences. The device 110 broadly comprises the body 112 and the two or more legs of either or both leg embodiments. The body 112 includes the reservoir 116 for containing the scent. An absorbent carrier 124, such as, for example, felt, cotton, or some other suitable natural or synthetic material, is retained within the body 112. One or more delivery tubes 126 extend from the reservoir 116 into the carrier 124, and present exit holes 128 adjacent to the carrier 124. The reservoir 116 is squeezable or otherwise actuatable to force the scent from the reservoir 116, through the delivery tubes 126, out the exit holes 128, and into the carrier 124 which absorbs the scent for subsequent release into the airflow. The reservoir 116 may include a port 130 for refilling the reservoir 116 with the scent. As an alternative to being manually squeezable or otherwise actuatable, the reservoir 116 may be automatically squeezable or otherwise actuatable, such as, for example, by a battery-powered actuating mechanism.

In a related embodiment, the reservoir is a trough fillable with scented fluid which automatically, such as, for example, by gravity, drips or otherwise flows downward onto or into the absorbent carrier, from which it is dispersed into the airflow.

From the preceding description, it will be appreciated that the scent delivery device of the present invention provides a number of substantial advantages over the prior art, including, for example, allowing for the wide dispersal of scent even when artificial airflow generators, such as heating and cooling apparatuses, have been turned off.

Although the invention has been described with reference to the preferred embodiments illustrated in the attached drawings, it is noted that equivalents may be employed and substitutions made herein without departing from the scope of the invention as recited in the claims.

Having thus described the preferred embodiment of the invention, what is claimed as new and desired to be protected by Letters Patent includes the following:

1. A scent delivery device for attachment to a door or window screen exposed to a natural airflow for dispersing a scent over time, the device comprising:
a body including
a reservoir for containing the scent, the reservoir being exposed to the natural airflow, and
an opening through which the natural airflow can flow; and
two or more legs connected to and extending outwardly from the body for attaching the body to the screen.

2. The scent delivery device as set forth in claim 1, further including a movable panel for controlling a size of the opening.

3. The scent delivery device as set forth in claim 1, wherein the reservoir is refillable with the scent.

4. The scent delivery device as set forth in claim 1, wherein the scent is in a substantially liquid form.

5. The scent delivery device as set forth in claim 4, further including a substantially solid carrier for receiving and releasing the scent over time into the natural airflow.

6. The scent delivery device as set forth in claim 1, wherein the two or more legs each include a length of wire having a diameter which is smaller than the mesh of the screen.

7. The scent delivery device as set forth in claim 6, wherein at least one of the two or more legs is bendable so as to positively engage the screen.

8. The scent delivery device as set forth in claim 6, wherein at least one of the two or more legs has the shape of a hook to positively engage the screen.

9. The scent delivery device as set forth in claim 1, wherein the body has substantially the shape of an insect.

10. The scent delivery device as set forth in claim 1, wherein the body has substantially the shape of a leaf.

11. The scent delivery device as set forth in claim 1, further including an absorbent carrier for receiving the scent from the reservoir.

12. The scent delivery device as set forth in claim 11, wherein the reservoir is actuatable to force the scent through delivery tubes and into the absorbent carrier

13. A scent delivery device for attachment to a door or window screen exposed to a natural airflow for dispersing a scent over time, the device comprising:
a body including
a reservoir for containing the scent, the reservoir being exposed to the natural airflow,
an opening through which the natural airflow can flow, and
a movable panel for controlling a size of the opening; and
two or more legs connected to and extending outwardly from the body for attaching the body to the screen, the two or more legs each including a length of wire having a diameter which is smaller than the mesh of the screen.

14. The scent delivery device as set forth in claim 13, wherein the scent is in a substantially liquid form, and further including a substantially solid carrier for receiving and releasing the scent over time into the natural airflow.

15. The scent delivery device as set forth in claim 13, wherein at least one of the two or more legs is bendable so as to positively engage the screen.

16. The scent delivery device as set forth in claim 13, wherein at least one of the two or more legs has the shape of a hook to positively engage the screen.

17. A scent delivery device for attachment to a door or window screen exposed to a natural airflow for dispersing a scent over time, the device comprising:
a body including
an actuatable reservoir for holding the scent,
an absorbent carrier for receiving the scent from the reservoir,
a delivery tube extending between the actuatable reservoir and the absorbent carrier for delivering the scent from the actuatable reservoir to the absorbent carrier when the actuatable reservoir is actuated, and
an opening through which the natural airflow can flow; and
two or more legs connected to and extending outwardly from the body for attaching the body to the screen.
